(19) United States
(12) Patent Application Publication
(10) Pub. No.: US 2009/0054858 A1
(43) Pub. Date: Feb. 26, 2009

(54) Layered sanitary tissue product having trichomes

(76) Inventors: Wendy Da Wei Cheng, Mason, OH (US); Kenneth Douglas Vinson, Toone, TN (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/894,519

(22) Filed: Aug. 21, 2007

(51) Int. Cl. A61F 13/15

(52) U.S. Cl. 604/358

(57) ABSTRACT

A layered fibrous structure having a machine direction, cross machine direction, and Z-direction. The fibrous structure also has a consumer side and first and second layers in the Z-direction where the first layer has a plurality of trichomes. The trichomes make up greater than about 0.1%, by weight, of the fibrous structure.

### FIELD OF THE INVENTION

The present invention relates to absorbent paper products. In particular, the present invention relates to sanitary tissue products comprising trichomes.

### BACKGROUND OF THE INVENTION

By and large, consumers of family and household care paper products prefer such products to feel soft and at the same time prefer such products to be strong so that the products can withstand the rigor of use.

Without wishing to be limited by theory, it is thought that the softness and strength of a tissue paper web are inversely related. In other words, the stronger a paper product is, the less soft the product is likely to be when compared to a paper product with a lower strength. Through the use of technology such as synthetic fibers, chemical softeners, or certain papermaking and/or converting methods, it is possible to increase both the softness and the strength of a tissue paper web. Such technology can work very well, but can be somewhat expensive and can sometimes result in a more expensive product to the consumer. As a result, the use of natural fibers is highly desirable because, without being limited by theory, it is thought that natural fibers provide the benefits of being more cost effective, are relatively easy to obtain, and provide a greater variety of fibers to choose from than synthetic materials.

Accordingly, it is an object of the present invention to provide a sanitary tissue paper product comprising natural fibers, such as trichomes, that provide increased tensile strength that does not detrimentally affect the softness of the paper, and in some embodiments, increase softness.

### SUMMARY OF THE INVENTION

In one embodiment the present invention relates to a layered fibrous structure comprising a machine direction, cross machine direction, and Z-direction. The fibrous structure further comprises a consumer side, and first and second layers in the Z-direction. The first layer comprises a plurality of trichomes wherein the trichomes comprise greater than about 0.1% of the fibrous structure by weight.

In another embodiment the present invention relates to a layered fibrous structure comprising a machine direction, cross machine direction, and Z-direction. The fibrous structure further comprises a consumer side, and first and second layers in the Z-direction. The first layer comprises a plurality of trichomes wherein the trichomes comprise greater than about 0.1% of the fibrous structure by weight. The fibrous structure further comprises a total dry tensile strength, the total dry tensile strength being at least about 10% greater than the total dry tensile strength of a comparable non-trichome ply.

In yet another embodiment, the present invention relates to a method for making a layered fibrous structure comprising two or more layers wherein at least one of the layers comprises a plurality of trichomes comprising the steps of: (a) preparing a first fiber furnish comprising a plurality of trichomes; (b) preparing a second fiber furnish; (c) combining the first fiber furnish and the second fiber furnish to provide a third fiber furnish; (d) depositing the third fiber furnish on a forming surface to form an embryonic fibrous web; and (e) drying the embryonic fibrous web.

### BRIEF DESCRIPTION OF THE DRAWINGS

FIG. 1 is a schematic view of an exemplary papermaking machine.

FIG. 2 is an enlarged schematic view of a Yankee Dryer and Creping Blade as shown in FIG. 1.

FIG. 3 is an exemplary embodiment of a cross-sectional view of a fibrous structure according to the present invention.

FIG. 4 is an exemplary embodiment of a cross-sectional view of a fibrous structure according to the present invention.

### DETAILED DESCRIPTION OF THE INVENTION

### Definitions

“Trichome,” as used herein, means an epidermal attachment of a varying shape, structure and/or function of a non-seed portion of a plant. In one example, a trichome is an outgrowth of the epidermis of a non-seed portion of a plant. The outgrowth may extend from an epidermal cell. In one embodiment, the outgrowth is a trichome fiber. The outgrowth may be a hairlike or bristlelike outgrowth from the epidermis of a plant.

Trichomes may be glandular or non-glandular. Glandular trichomes have active secretory capability; glandular trichomes may, for example, secrete oil, resin or mucilage. A typical glandular trichome possesses a stalk and enlarged terminal portion, which may be referred to as gland. Active secretory cells of glandular trichomes have dense protoplasts and elaborate various substances, such as volatile oil, resin and mucilage. Non-glandular trichomes are typically unicellular or multicellular fiber-like in nature and substantially free of any active secretion capability or other impurities, although they may contain minor amounts of impurities which may be extracted by water or other solvents.

Trichomes may protect the plant tissues present on a plant. Trichomes may for example protect leaves and stems from attack by other organisms, particularly insects or other foraging animals and/or they may regulate light and/or temperature and/or moisture. They may also produce glands in the forms of scales, different papills and, in roots, the sometimes function to absorb water and/or moisture. A trichome may be formed by one cell or by a plurality of cells.

“Individualized trichome,” as used herein, means trichomes which have been artificially separated by a suitable method for individualizing trichomes from their host plant. In other words, individualized trichomes, as used herein, means that the trichomes become separated from a non-seed portion of a host plant by some non-naturally occurring action. Primarily, individualized trichomes will be fragments or entire trichomes with essentially no remnant of the host plant attached. However, individualized trichomes can also comprise a minor fraction of trichomes retaining a portion of the host plant still attached, as well as a minor fraction of trichomes in the form of a plurality of trichomes bound by their individual attachment to a common remnant of the host plant. Individualized trichomes may comprise a portion of a pulp or mass further comprising other materials. Other materials include non-trichome-bearing fragments of the host plant.

In one example of the present invention, the individualized trichomes may be classified to enrich the individualized trichomal content at the expense of mass not constituting individualized trichomes.

Individualized trichomes may be converted into chemical derivatives including but not limited to cellulose derivatives, for example, regenerated cellulose such as rayon; cellulose ethers such as methyl cellulose, carboxymethyl cellulose, and hydroxyethyl cellulose; cellulose esters such as cellulose acetate and cellulose butyrate; and nitrocellulose. Individualized trichomes may also be used in their physical form, usually fibrous, and herein referred to “trichome fibers”, as a component of fibrous structures.

Trichome fibers are different from seed hair fibers in that they are not attached to seed portions of a plant. For example, trichome fibers, unlike seed hair fibers, are not attached to a seed or a seed pod epidermis. Cotton, kapok, milkweed, and coconut coir are nonlimiting examples of seed hair fibers.

Further, trichome fibers are different from nonwood bast and/or core fibers in that they are not attached to the bast, also known as phloem, or the core, also known as xylem portions of a nonwood dicotyledonous plant stem. Nonlimiting examples of plants which have been used to yield nonwood bast fibers and/or nonwood core fibers include kenaf, jute, flax, ramie and hemp.

Trichome fibers are different from leaf fibers in that they do not originate from within the leaf structure. Sisal and abaca are sometimes liberated as leaf fibers.

In addition, trichome fibers are different from monocotyledonous plant derived fibers such as those derived from cereal straws (wheat, rye, barley, oat, etc), stalks (corn, cotton, sorghum, Hesperaloe funifera, etc.), canes (bamboo, bagasse, etc.), grasses (esparto, lemon, sabai, switchgrass, etc), since such monocotyledonous plant derived fibers are not attached to an epidermis of a plant.

Finally, trichome fibers are different from wood pulp fibers since wood pulp fibers are not outgrowths from the epidermis of a plant; namely, a tree. Wood pulp fibers rather originate from the secondary xylem portion of the tree stem.

“Fiber,” as used herein, means an elongate physical structure having an apparent length greatly exceeding its apparent diameter, i.e. a length to diameter ratio of at least about 10. Fibers having a non-circular cross-section and/or tubular shape are common; the “diameter” in this case may be considered to be the diameter of a circle having cross-sectional area equal to the cross-sectional area of the fiber. More specifically, as used herein, “fiber” refers to fibrous structure-making fibers. The present invention contemplates the use of a variety of fibrous structure-making fibers, such as, for example, natural fibers or synthetic fibers, or any other suitable fibers, and any combination thereof.

Natural fibers useful in the present invention include animal fibers, mineral fibers, other plant fibers (in addition to the trichomes of the present invention) and mixtures thereof. Animal fibers may, for example, be selected from the group consisting of: wool, silk and mixtures thereof. The other plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers, which may be known to those of skill in the art as wood pulps, include chemical pulps, such as Kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered web and are described in more detail in U.S. Pat. Nos. 4,300,981 and 3,994,771. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. In one example, the fibers are softwood fibers derived from the kraft process and originating from more-northern climates. These are often referred to as northern softwood kraft (NSK) pulps.

Synthetic fibers may be selected from the group consisting of: wet spun fibers, dry spun fibers, melt spun (including melt blown) fibers, synthetic pulp fibers and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

The web (fibrous structure) of the present invention may comprise fibers, films and/or foams that comprise a hydroxyl polymer and optionally a crosslinking system. Nonlimiting examples of suitable hydroxyl polymers include polyols, such as polyvinyl alcohol, polyvinyl alcohol derivatives, polyvinyl alcohol copolymers, starch, starch derivatives, chitosan, chitosan derivatives, cellulose derivatives such as cellulose ether and ester derivatives, gums, arabinans, galactans, proteins and various other polysaccharides and mixtures thereof. For example, a web of the present invention may comprise a continuous or substantially continuous fiber comprising a starch hydroxyl polymer and a polyvinyl alcohol hydroxyl polymer produced by dry spinning and/or solvent spinning (both unlike wet spinning into a coagulating bath) a composition comprising the starch hydroxyl polymer and the polyvinyl alcohol hydroxyl polymer.

“Fibrous structure,” as used herein, means a structure that comprises one or more fibers. In one example, a fibrous structure according to the present invention means an orderly arrangement of fibers within a structure in order to perform a function. Nonlimiting examples of fibrous structures of the present invention include composite materials (including reinforced plastics and reinforced cement), paper, fabrics (including woven, knitted, and non-woven), and absorbent pads (for example for diapers or feminine hygiene products). A bag of loose fibers is not a fibrous structure in accordance with the present invention.

Nonlimiting examples of processes for making fibrous structures include known wet-laid papermaking processes and air-laid papermaking processes. Such processes typically include steps of preparing a fiber composition in the form of a suspension in a medium, either wet, more specifically aqueous medium, or dry, more specifically gaseous, i.e. with air as medium. The aqueous medium used for wet-laid processes is oftentimes referred to as a fiber slurry. The fibrous suspension is then used to deposit a plurality of fibers onto a forming wire or belt such that an embryonic fibrous structure is formed, after which drying and/or bonding the fibers together results in a fibrous structure. Further processing the fibrous structure may be carried out such that a finished fibrous structure is formed. For example, in typical papermaking processes, the finished fibrous structure is the fibrous structure that is wound on the reel at the end of papermaking, and may subsequently be converted into a finished product, e.g. a sanitary tissue product.

“Sanitary tissue product” or “tissue product,” as used herein, means a fibrous structure product comprising one or more finished fibrous structures that may, or may not be, converted. In one embodiment, the sanitary tissue product that is useful as a wiping implement for post-urinary and post-bowel movement cleaning (toilet tissue), for otorhinolaryngological discharges (facial tissue), and multi-functional absorbent and cleaning uses (absorbent towels).

“Dry Tensile Strength” sometimes known to those of skill in the art as “Tensile Strength” of a fibrous structure, as used herein, is measured as follows: One (1) inch by five (5) inch (2.5 cm×12.7 cm) strips of fibrous structure and/or paper product comprising such fibrous structure are provided. The strip is placed on an electronic tensile tester Model 1122 commercially available from Instron Corp., Canton, Mass. in a conditioned room at a temperature of 73° F.±4° F. (about 28° C.±2.2° C.) and a relative humidity of 50%±10%. The crosshead speed of the tensile tester is 2.0 inches per minute (about 5.1 cm/minute) and the gauge length is 4.0 inches (about 10.2 cm). The Dry Tensile Strength can be measured in any direction by this method. The “Total Dry Tensile Strength” or “TDT” is the special case determined by the arithmetic total of MD and CD tensile strengths of the strips.

“Modulus” or “Tensile Modulus,” as used herein, means the slope tangent to the load elongation curve taken at the point corresponding to 15 g/cm-width upon conducting a tensile measurement as specified in the foregoing.

“Layers,” as used herein, means a tier comprising papermaking fibers having some thickness in the Z-direction. Without wishing to be limited by theory it is thought that different furnishes can be supplied from different sources to provide a fibrous structure having layers. Each furnish can comprise any combination of fibers, natural or synthetic as discussed supra. Also without wishing to be limited by theory it is thought that the layers are not completely discrete and that there is some intermixing of components between the different layers at the boundary between two layers.

“Comparable Non-Trichome Ply,” or “Comparable Non-Trichome Fibrous Structure,” as used herein, refers to a so-called “beta” ply of fibrous structure that does not contain trichomes, but otherwise comprises substantially the same composition (fibers and other papermaking materials, layers, and the like) as a so-called “alpha” ply of fibrous structure that comprises trichomes. The beta (comparable non-trichome) ply comprises non-trichome fibers in the same weight as the alpha ply has in trichomes in the analogous layer(s) as the alpha ply. For example, an alpha ply comprises a first layer and a second layer wherein the first layer comprises 2% by weight of the entire ply of trichomes and the remainder of the first layer comprises eucalyptus. The comparable beta (non-trichome) ply to the aforementioned alpha (trichome) ply comprises a first layer and a second layer wherein the second layer of the comparable beta ply is identical to the second layer of the alpha ply. The first layer of the beta ply comprises eucalyptus only because the first layer of the alpha ply comprises eucalyptus only.

“Consumer Side,” as used herein, means the surface of a sanitary tissue paper that the consumer will touch when using. In a multi-ply sanitary tissue paper product, the consumer sides are the two outward facing surfaces.

“Machine Direction” or “MD,” as used herein, means the direction parallel to the flow of the fibrous structure and/or paper web through a papermaking machine and/or product manufacturing equipment.

“Cross Machine Direction” or “CD,” as used herein, means the direction perpendicular to, and coplanar with, the machine direction of the fibrous structure and/or paper web.

“Z-direction,” as used herein, means the direction normal to a plane formed by machine direction and cross machine directions.

### Trichomes

Without wishing to be limited by theory, it is thought that almost all plants have trichomes. Those skilled in the art may recognize that some plants will have trichomes of sufficient mass fraction and/or the overall growth rate and/or robustness of the plant so that they may offer attractive agricultural economy to make them more suitable for a large commercial process, such as using them as a source of chemicals, e.g. cellulose, or assembling them into fibrous structures, such as disposable fibrous structures. Trichomes may have a wide range of morphology and chemical properties. For example, the trichomes may be in the form of fibers; namely, trichome fibers. Such trichome fibers may have a length to diameter ratio that is greater than 1. In one example of the present invention, the trichome is a non-glandular trichome.

Nonlimiting examples of suitable sources for obtaining trichomes, especially trichome fibers, are plants in the Labiatae (Lamiaceae) family commonly referred to as the mint family.

The following sources are offered as nonlimiting examples of trichome-bearing plants (suitable sources) for obtaining trichomes, especially trichome fibers.

Examples of suitable species in the Labiatae family include Stachys byzantina, also known as Stachys lanata commonly referred to as lamb's ear, woolly betony, or woundwort. The term Stachys byzantina as used herein also includes cultivars Stachys byzantina ‘Primrose Heron’, Stachys byzantina ‘Helene von Stein’ (sometimes referred to as Stachys byzantina ‘Big Ears’), Stachys byzantina ‘Cotton Boll’, Stachys byzantina ‘Variegated’ (sometimes referred to as Stachys byzantina ‘Striped Phantom’), and Stachys byzantina ‘Silver Carpet’.

Additional examples of suitable species in the Labiatae family include the arcticus subspecies of Thymus praecox, commonly referred to as creeping thyme and the pseudolanuginosus subspecies of Thymus praecox, commonly referred to as wooly thyme.

Further examples of suitable species in the Labiatae family include several species in the genus Salvia (sage), including Salvia leucantha, commonly referred to as the Mexican bush sage; Salvia tarahumara, commonly referred to as the grape scented Indian sage; Salvia apiana, commonly referred to as white sage; Salvia funereal, commonly referred to as Death Valley sage; Salvia sagittata, commonly referred to as balsamic sage; and Salvia argentiae, commonly referred to as silver sage.

Even further examples of suitable species in the Labiatae family include Lavandula lanata, commonly referred to as wooly lavender; Marrubium vulgare, commonly referred to as horehound; Plectranthus argentatus, commonly referred to as silver shield; i Plectranthus tomentosa.

Nonlimiting examples of other suitable sources for obtaining trichomes, especially trichome fibers are plants in the Asteraceae family commonly referred to as the sunflower family.

Examples of suitable species in the Asteraceae family include Artemisia stelleriana, also known as silver brocade; Haplopappus macronema, also known as the whitestem goldenbush; Helichrysum petiolare; Centaurea maritime, also known as Centaurea gymnocarpa or dusty miller; Achillea tomentosum, also known as wooly yarrow; Anaphalis margaritacea, also known as pearly everlasting; and Encelia farinose, also known as brittle bush.

Additional examples of suitable species in the Asteraceae family include Senecio brachyglottis and Senecio haworthii, the latter also known as Kleinia haworthii.

Nonlimiting examples of other suitable sources for obtaining trichomes, especially trichome fibers, are plants in the Scrophulariaceae family commonly referred to as the figwort or snapdragon family.

An example of a suitable species in the Scrophulariaceae family includes Pedicularis kanei, also known as the wooly lousewort.

Additional examples of suitable species in the Scrophulariaceae family include the mullein species (Verbascum) such as Verbascum hybridium, also known as snow maiden; Verbascum thapsus, also known as common mullein; Verbascum baldaccii; Verbascum bombyciferum; Verbascum broussa; Verbascum chaixii; Verbascum dumulsum; Verbascum laciniatum; Verbascum lanatum; Verbascum longifolium; Verbascum lychnitis; Verbascum olympicum; Verbascum paniculatum, Verbascum phlomoides, Verbascum phoeniceum; Verbascum speciosum, Verbascum thapsiforme; Verbascum virgatum; Verbascum wiedemannianum; and various mullein hybrids including Verbascum ‘Helen Johnson’ and Verbascum ‘Jackie’.

Further examples of suitable species in the Scrophulariaceae family include Stemodia tomentosa and Stemodia durantifolia.

Nonlimiting examples of other suitable sources for obtaining trichomes, especially trichome fibers include Greyia radlkoferi and Greyia flanmaganii plants in the Greyiaceae family commonly referred to as the wild bottlebrush family.

Nonlimiting examples of other suitable sources for obtaining trichomes, especially trichome fibers include members of the Fabaceae (legume) family. These include the Glycine max, commonly referred to as the soybean, and Trifolium pratense L, commonly referred to as medium and/or mammoth red clover.

Nonlimiting examples of other suitable sources for obtaining trichomes, especially trichome fibers include members of the Solanaceae family including varieties of Lycopersicum esculentum, otherwise known as the common tomato.

Nonlimiting examples of other suitable sources for obtaining trichomes, especially trichome fibers include members of the Convolvulaceae (morning glory) family, including Argyreia nervosa, commonly referred to as the wooly morning glory and Convolvulus cneorum, commonly referred to as the bush morning glory.

Nonlimiting examples of other suitable sources for obtaining trichomes, especially trichome fibers include members of the Malvaceae (mallow) family, including Anoda cristata, commonly referred to as spurred anoda and Abutilon theophrasti, commonly referred to as velvetleaf.

Nonlimiting examples of other suitable sources for obtaining trichomes, especially trichome fibers include Buddleia marrubiifolia, commonly referred to as the wooly butterfly bush of the Loganiaceae family; the Casimiroa tetrameria, commonly referred to as the wooly leafed sapote of the Rutaceae family; the Ceanothus tomentosus, commonly referred to as the wooly leafed mountain liliac of the Rhamnaceae family; the ‘Philippe Vapelle’ cultivar of renardii in the Geraniaceae (geranium) family; the Tibouchina urvilleana, commonly referred to as the Brazilian spider flower of the Melastomataceae family; the Tillandsia recurvata, commonly referred to as ballmoss of the Bromeliaceae (pineapple) family; the Hypericum tomentosum, commonly referred to as the wooly St. John's wort of the Hypericaceae family; the Chorizanthe orcuttiana, commonly referred to as the San Diego spineflower of the Polygonaceae family; Eremocarpus setigerus, commonly referred to as the doveweed of the Euphorbiaceae or spurge family; Kalanchoe tomentosa, commonly referred to as the panda plant of the Crassulaceae family; and Cynodon dactylon, commonly referred to as Bermuda grass, of the Poaceae family; and Congea tomentosa, commonly referred to as the shower orchid, of the Verbenaceae family.

Suitable trichome-bearing plants are commercially available from nurseries and other plant-selling commercial venues. For example, Stachys byzantina may be purchased and/or viewed at Blanchette Gardens, Carlisle, Mass. In one embodiment a trichome suitable for use in the fibrous structures of the present invention comprises cellulose.

### Individualized Trichomes

Trichomes may be obtained from suitable plant sources by any suitable method known in the art. Nonlimiting examples of suitable methods include the step of separating a trichome from an epidermis of a non-seed portion of a plant. In some embodiments, trichomes may be individualized using mechanical and/or chemical process steps.

Nonlimiting examples of mechanical process steps include contacting an epidermis of a non-seed portion of a trichome-bearing plant with a device such that a trichome is separated from the epidermis. Nonlimiting examples of such devices for use in such a contacting step include a ball mill, a pin mill, a hammermill, a rotary knife cutter such as a “Wiley Mill” and/or a “CoMil” sold by Quadro Engineering of Waterloo, Ontario, Canada.

In one example, an epidermis of a non-seed portion of a trichome-bearing plant is subjected to a mill device that comprises a screen, in particular, a slotted screen, designed to better separate the trichome-bearing material from the plant epidermis.

The trichome-bearing material may be subjected to a mechanical process to liberate its trichomes from its plant epidermis to enrich the pulp or fiber mass' content of individualized trichomes. This may be carried out by means of screening or air classifying equipment well known in the art. A suitable air classifier is the Hosokawa Alpine 50ATP, sold by Hosokawa Micron Powder Systems of Summit, N.J. In one example, the pulp or fiber mass' content of the individualized trichomes is subjected to one or more air classifying steps and then the pulp or fiber mass remaining after the air classifying step(s) is subjected to one or more screeners to further enrich the pulp or fiber mass' content of individualized trichomes.

Alternatively, the creation of individualized trichomes may employ wet processes practiced on the trichome bearing plant, optionally in combination with mechanical treatment. This includes processes analogous to the well known (in the wood pulp industry) groundwood, refiner-mechanical pulping, or thermo-mechanical pulping means, followed optionally by wet classification to enrich the individualized trichomes. Wet processes also include chemical processes, nonlimiting examples of which include contacting an epidermis of a non-seed portion of a trichome-bearing plant with a chemical composition such that a trichome is separated from the epidermis. Suitable chemical process steps include the chemical process steps of the well-known (in the wood pulp industry) Kraft, sulfite and/or soda processes, including chemi-mechanical variations.

Further examples of trichomes and processes for individualizing trichomes are discussed in detail in U.S. patent application Ser. No. 11/436,494.

### Fibrous Structures

The fibrous structures of the present invention may comprise a trichome, especially a trichome fiber, or a plurality of trichomes. In addition to a trichome, other fibers and/or other ingredients may also be present in the fibrous structures of the present invention.

Nonlimiting types of fibrous structures according to the present invention include conventionally felt-pressed fibrous structures; pattern densified fibrous structures; and high-bulk, uncompacted fibrous structures. In some embodiments, the fibrous structures may be of a homogenous or multilayered (two or three or more layers) construction; and the sanitary tissue products made therefrom may be of a single-ply or multi-ply construction.

In one example, the fibrous structure of the present invention is a pattern densified fibrous structure characterized by having a relatively high-bulk region of relatively low fiber density and an array of densified regions of relatively high fiber density. The high-bulk field is characterized as a field of pillow regions. The densified zones are referred to as knuckle regions. The knuckle regions exhibit greater density than the pillow regions. The densified zones may be discretely spaced within the high-bulk field or may be interconnected, either fully or partially, within the high-bulk field. Typically from about 8% to about 65% of the fibrous structure surface comprises densified knuckles, the knuckles may exhibit a relative density of at least 125% of the density of the high-bulk field. Processes for making pattern densified fibrous structures are well known in the art as exemplified in U.S. Pat. Nos. 3,301,746, 3,974,025, 4,191,609 and 4,637,859.

The fibrous structures comprising a trichome in accordance with the present invention may be in the form of through-air-dried fibrous structures, differential density fibrous structures, differential basis weight fibrous structures, wet laid fibrous structures, air laid fibrous structures (examples of which are described in U.S. Pat. Nos. 3,949,035 and 3,825,381), conventional dried fibrous structures, creped or uncreped fibrous structures, patterned-densified or non-patterned-densified fibrous structures, compacted or uncompacted fibrous structures, nonwoven fibrous structures comprising synthetic or multicomponent fibers, homogeneous or multilayered fibrous structures, double re-creped fibrous structures, foreshortened fibrous structures, co-form fibrous structures (examples of which are described in U.S. Pat. No. 4,100,324) and mixtures thereof.

In one example, the air laid fibrous structure is selected from the group consisting of thermal bonded air laid (TBAL) fibrous structures, latex bonded air laid (LBAL) fibrous structures and mixed bonded air laid (MBAL) fibrous structures.

The fibrous structures may exhibit a substantially uniform density or may exhibit differential density regions, in other words regions of high density compared to other regions within the patterned fibrous structure. Typically, when a fibrous structure is not pressed against a cylindrical dryer, such as a Yankee dryer, while the fibrous structure is still wet and supported by a through-air-drying fabric or by another fabric or when an air laid fibrous structure is not spot bonded, the fibrous structure typically exhibits a substantially uniform density.

In addition to a trichome, the fibrous structure may comprise other additives, such as wet strength additives, softening additives, solid additives (such as starch, clays), dry strength resins, wetting agents, lint resisting agents, absorbency-enhancing agents, immobilizing agents, especially in combination with emollient lotion compositions, antiviral agents including organic acids, antibacterial agents, polyol polyesters, antimigration agents, polyhydroxy plasticizers and mixtures thereof. Such other additives may be added to the fiber furnish, the embryonic fibrous web and/or the fibrous structure. Such other additives may be present in the fibrous structure at any level based on the dry weight of the fibrous structure. In some embodiments, other additives may be present in the fibrous structure at a level of from about 0.001 to about 50% and/or from about 0.001 to about 20% and/or from about 0.01 to about 5% and/or from about 0.03 to about 3% and/or from about 0.1 to about 1.0% by weight, on a dry fibrous structure basis.

The fibrous structures of the present invention may be subjected to any suitable post processing including, but not limited to, printing, embossing, calendaring, slitting, folding, combining with other fibrous structures, and the like.

### Papermaking Machine/Processes for Making Trichome-Containing Fibrous Structures

Any suitable process for making fibrous structures known in the art may be used to make trichome-containing fibrous structures of the present invention. In one embodiment the trichome-containing fibrous structures of the present invention can be made by a wet laid fibrous structure making process. In another embodiment the trichome-containing fibrous structures of the present invention can be made by an air laid fibrous structure making process.

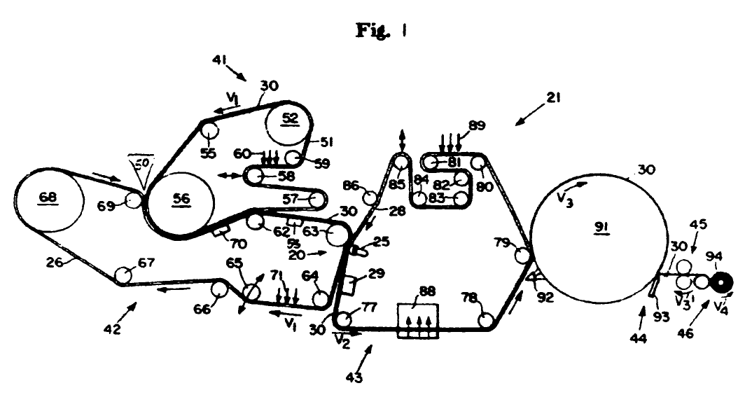

FIG. 1 shows a schematic view of an exemplary papermaking machine 21 in which the present invention fibrous structures may be made. The papermaking machine 21 comprises transfer zone 20 as described herein and, additionally: a forming section 41, an intermediate carrier section 42, a pre-dryer/imprinting section 43, a drying/creping section 44, a calendar assembly 45, and reel 46.

The forming section 41 of the papermaking machine 21 comprises a headbox 50; a loop of fine mesh backing wire or fabric 51 which is looped about a vacuum breast roll 52, over vacuum box 70, about rolls 55 through 59, and under showers 60. Fabric 51 is deflected from a straight run by intermediate rolls 56 and 57 and a separation roll 62. Biasing means not shown are provided for moving roll 58 as indicated by the adjacent arrow to maintain fabric 51 in a slack obviating tensioned state.

The intermediate carrier section 42 comprises a loop of forming and carrier fabric 26 which is looped about rolls 62 through 69 and about a portion of roll 56. The forming and carrier fabric 26 also passes over vacuum boxes 70 and 53, and transfer head 25; and under showers 71. Biasing means are also provided to move roll 65 to obviate slack in carrier fabric 26. Juxtaposed portions of fabrics 51 and 26 extend about an arcuate portion of roll 56, across vacuum box 70, and separate after passing over an arcuate portion of separation roll 62. In one embodiment, carrier fabric 26 is identical to backing fabric 51 except for the length.

The pre-dryer/imprinting section 43 of papermaking machine 21 comprises a loop of transfer fabric or imprinting fabric 28. Transfer/imprinting fabric 28 is looped about rolls 77 through 86; passes across transfer head 25 and vacuum box 29; through a blow-through pre-dryer 88; and under showers 89. In the exemplary embodiment a biasing roll 79 set at a predetermined force per lineal inch may effect imprinting a knuckle pattern of the transfer/imprinting fabric 28 into a fibrous structure 30 in the manner of, and for the purpose disclosed in, U.S. Pat. No. 3,301,746.

The drying/creping section 44 of papermaking machine 21 comprises Yankee dryer 91, adhesive applicator 92, creping blade 93, and reel roll 94.

V₁ is the velocity of the papermaking fabrics 51 and 26. V₂ is the velocity about the transfer/printing rolls 77 through 86. V₃ is the velocity of the calendar assembly 45. V₄ is the reel velocity of the reel roll 94.

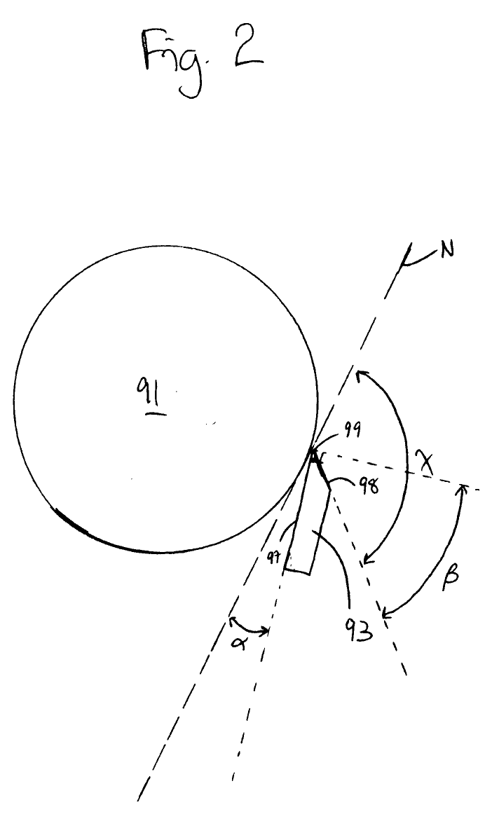

FIG. 2 shows an enlarged schematic view of the Yankee dryer 91 and creping blade 93 shown in FIG. 1. Line N shows a line that is tangent to the surface of the Yankee dryer 91 which passes through the point of contact between the Yankee dryer 91 and the creping blade 93. The creping angle α is the angle that is formed between the leading side 97 of the creping blade 93 and N. The impact angle χ is the angle that is formed between the bevel surface 98 of the creping blade 93 and N. In one embodiment χ is from about 70 degrees to about 100 degrees. In another embodiment χ is from about 80 degrees to about 90 degrees. The blade bevel angle β is the angle that is measured between line T (the line that is perpendicular to the leading side 97 of the creping blade 93 at the tip 99 of the creping blade 93) and the bevel surface 98 of the creping blade 93. In one embodiment β is greater than about 65 degrees. In another embodiment β is from about 65 degrees to about 90 degrees

In one embodiment a trichome-containing fibrous structure can be made by the process comprising the steps of: a) preparing a first fiber furnish (slurry) comprising a plurality of trichomes; b) preparing a second fiber furnish (slurry); c) combining the first fiber furnish and the second fiber furnish in a pipe to provide a third fiber furnish; d) depositing the third fiber furnish on a forming fabric 26 to form an embryonic fibrous web; and e) drying the embryonic fibrous web to form a trichome-containing fibrous structure. In another embodiment the process further comprises the steps of: f) preparing a fourth fiber furnish, g) preparing a fifth fiber furnish, and h) depositing the fourth and fifth fiber furnishes on the forming surface 26 prior to drying to form an embryonic fibrous web having multiple layers. It is surprisingly found that combining the first and second fiber furnishes in a pipe provides a fibrous structure with an unexpectedly high total dry tensile strength. In one embodiment the second fiber furnish comprises Eucalyptus fibers. In some embodiments, the fourth and/or fifth furnishes may comprise any fiber (natural or synthetic) discussed supra. In one embodiment the fourth fiber furnish comprises Northern Softwood Kraft (NSK) fibers. In one embodiment, the fifth fiber furnish further comprises Eucalyptus fibers. In one embodiment, one or more fiber furnishes may be deposited onto the foraminous forming surface via a headbox.

In one embodiment, the process further comprises the steps of: i) drying the embryonic fibrous web on a Yankee dryer 91 to form a trichome-containing fibrous structure, and j) optionally creping the trichome containing fibrous structure with a creping blade 93. In another embodiment, for example, the embryonic fibrous web is dried such that the trichome-containing (in one embodiment, the third) fiber furnish contacts the drying roll during drying.

### Layered Ply

The trichome-containing fibrous structure of the present invention may comprise two or more layers in the Z-direction. Layers are described in greater detail, for example, in U.S. Pat. No. 4,300,981. Without wishing to be limited by theory, it is thought that the inclusion of trichomes in a layer of a ply of fibrous structure 30 (shown in FIG. 1) increases the total dry tensile strength of the tissue product without lowering, and in some embodiments increasing, the surface softness of the fibrous structure.

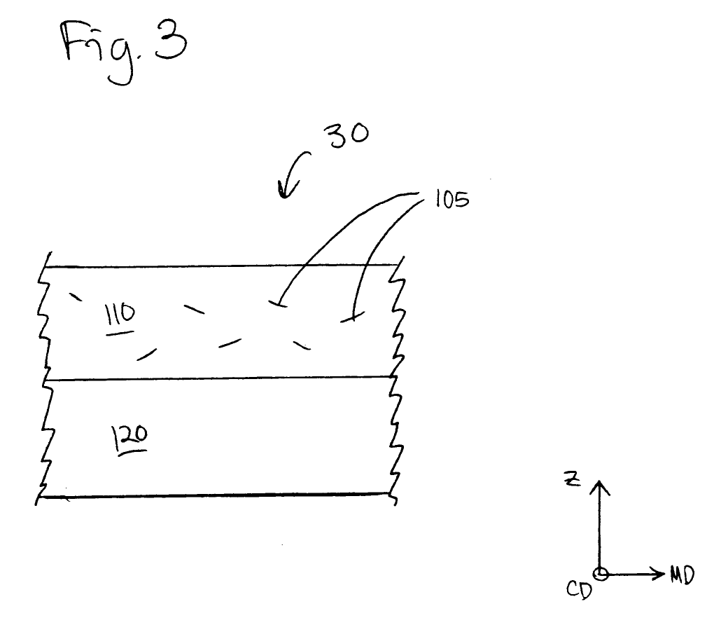

FIG. 3 shows a cross sectional view of an exemplary embodiment of a fibrous structure 30 of the present invention comprising a first layer 110 and a second layer 120. In some embodiments the first layer 110 comprises trichomes 105. In one embodiment the first layer 110 comprises greater than about 0.1% of trichomes 105 by weight of the fibrous structure 30. In another embodiment, the first layer 110 comprises from about 0.1% to about 15% trichomes 105 by weight of the fibrous structure 30. In another embodiment the first layer 110 comprises from about 1% to about 8% of trichomes 105 by weight of the fibrous structure 30. In another embodiment still the first layer 110 comprises from about 2% to about 4% of trichomes 105 by weight of the fibrous structure 30. In one embodiment, the first layer 110 comprises from about 5% to about 50% of the fibrous structure 30 in the Z-direction. In another embodiment the first layer 110 comprises from about 15% to about 35% of the fibrous structure 30 in the Z-direction.

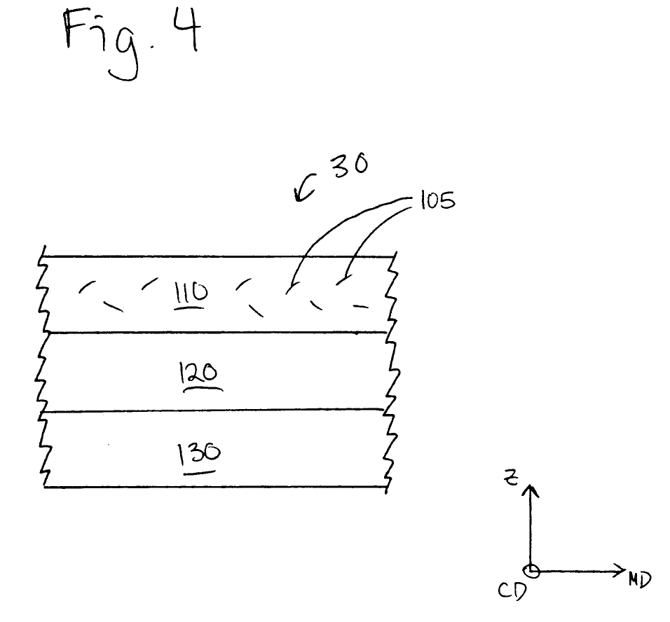

FIG. 4 shows a cross sectional view of an exemplary embodiment of a fibrous structure 30 of the present invention comprising a first layer 110, second layer 120 and a third layer 130. In one embodiment the first layer 110 comprises greater than about 0.1% of trichomes 105 by weight of the fibrous structure 30. In one embodiment the first layer 110 comprises from about 0.1% to about 15% trichomes 105 by weight of the fibrous structure 30. In another embodiment the first layer 110 comprises from about 1% to about 8% of trichomes 105 by weight of the fibrous structure 30. In another embodiment still the first layer 110 comprises from about 2% to about 4% of trichomes 105 by weight of the fibrous structure 30. In one embodiment the first layer comprises from about 5% to about 50% of the ply in the Z-direction. In one embodiment the second layer 120 comprises from about 15% to about 35% of the fibrous structure 30 in the Z-direction. In one embodiment the third layer 130 comprises from about 25% to about 70% of the fibrous structure 30 in the Z-direction. In one embodiment the first layer 110 comprises the remaining fibrous structure in the Z-direction. In one embodiment the first layer 110 is on the consumer side of the fibrous structure. In one embodiment, the trichomes 105 are individualized trichomes.

In one embodiment, the total dry tensile strength of a ply of a fibrous structure comprising two or more layers, wherein a first layer 110 comprises trichomes 105 as described supra, and has a total dry tensile strength of at least about 10% greater than the total dry tensile strength of the comparable non-trichome ply. In another embodiment the tensile strength of the trichome-containing ply is from about 10% to about 40% greater than the total dry tensile strength of the comparable non-trichome ply. In another embodiment the total dry tensile strength of the trichome-containing ply is from about 15% to about 30% greater than the total dry tensile strength of the comparable non-trichome ply.

### EXAMPLE 1

### Fibrous Structure without Trichomes

The following example illustrates a nonlimiting example for the preparation of a non-trichome containing fibrous structure on a pilot-scale Fourdrinier paper making machine.

An aqueous slurry of eucalyptus fibers is prepared at about 3% by weight using a conventional repulper. Separately, an aqueous slurry of NSK fibers of about 3% by weight is made up using a conventional repulper.

In order to impart temporary wet strength to the finished fibrous structure, a 1% dispersion of temporary wet strengthening additive (e.g., Parez.™. 750) is prepared and is added to the NSK fiber stock pipe at a rate sufficient to deliver 0.3% temporary wet strengthening additive based on the dry weight of the NSK fibers. The absorption of the temporary wet strengthening additive is enhanced by passing the treated slurry through an in-line mixer.

The eucalyptus fiber slurry is diluted with white water at the inlet of a fan pump to a consistency of about 0.15% based on the total weight of the eucalyptus fiber slurry. The NSK fibers, likewise, are diluted with white water at the inlet of a fan pump to a consistency of about 0.15% based on the total weight of the NSK fiber slurry. The eucalyptus fiber slurry and the NSK fiber slurry are both directed to a layered headbox capable of maintaining the slurries as separate streams until they are deposited onto a forming fabric on the Fourdrinier.

“DC 2310” (Dow Coming, Midland, Mich.) antifoam is dripped into the wirepit to control foam to maintain whitewater levels of 10 ppm.

The paper making machine has a layered headbox with a top chamber, a center chamber, and a bottom chamber. The eucalyptus fiber slurry is pumped through the top and bottom headbox chambers and, simultaneously, the NSK fiber slurry is pumped through the center headbox chamber and delivered in superposed relation onto a Fourdrinier wire to form thereon a three-layer embryonic web, of which about 70% is made up of the eucalyptus fibers and about 30% is made up of the NSK fibers. Dewatering occurs through the Fourdrinier wire and is assisted by a deflector and vacuum boxes. The Fourdrinier wire is of a 5-shed, satin weave configuration having 87 machine-direction and 76 cross-machine-direction monofilaments per inch, respectively. The speed of the Fourdrinier wire is about 750 fpm (feet per minute).

The embryonic wet web is transferred from the Fourdrinier wire, at a fiber consistency of about 15% at the point of transfer, to a patterned drying fabric. The speed of the patterned drying fabric is about the same as the speed of the Fourdrinier wire. The drying fabric is designed to yield a pattern densified tissue with discontinuous low-density deflected areas arranged within a continuous network of high density (knuckle) areas. This drying fabric is formed by casting an impervious resin surface onto a fiber mesh supporting fabric. The supporting fabric is a 98×62 filament, dual layer mesh. The thickness of the resin cast is about 12 mils above the supporting fabric. A suitable process for making the patterned drying fabric is described in published application US 2004/0084167 A1.

Further de-watering is accomplished by vacuum assisted drainage until the web has a fiber consistency of about 30%.

While remaining in contact with the patterned drying fabric, the web is pre-dried by air blow-through pre-dryers to a fiber consistency of about 65% by weight.

After the pre-dryers, the semi-dry web is transferred to the Yankee dryer and adhered to the surface of the Yankee dryer with a sprayed creping adhesive. The creping adhesive is an aqueous dispersion with the actives consisting of about 22% polyvinyl alcohol, about 11% CREPETROL A3025, and about 67% CREPETROL R6390. CREPETROL A3025 and CREPETROL R6390 are commercially available from Hercules Incorporated of Wilmington, Del. The creping adhesive is delivered to the Yankee surface at a rate of about 0.15% adhesive solids based on the dry weight of the web. The fiber consistency is increased to about 97% before the web is dry creped from the Yankee with a doctor blade.

The doctor blade has a bevel angle of about 25 degrees and is positioned with respect to the Yankee dryer to provide an impact angle of about 81 degrees. The Yankee dryer is operated at a temperature of about 350 Fahrenheit (about 177 Celsius) and a speed of about 800 fpm. The fibrous structure is wound in a roll using a surface driven reel drum having a surface speed of about 656 feet per minute. The fibrous structure may be subsequently converted into a two-ply sanitary tissue product having a basis weight of about 50 lbs/3000 ft².

The total dry tensile strength was measured as described supra. The resulting total dry tensile strength for the fibrous structure product having no trichomes is 328 g/cm.

### EXAMPLE 2

### Fibrous Structure with Trichomes

This following example illustrates a nonlimiting example for the preparation of a fibrous structure according to the present invention on a pilot-scale Fourdrinier paper making machine with the addition of trichomes providing a strength increase.

Individualized trichomes are first prepared from Stachys byzantina bloom stalks consisting of the dried stems, leaves, and pre-flowering buds, by passing dried Stachys byzantina plant matter through a knife cutter (Wiley mill, manufactured by the C. W. Brabender Co. located in South Hackensack, N.J.) equipped with an attrition screen having ¼″ holes. A composite fluff constituting the individualized trichome fibers together with chunks of leaf and stem material exits the Wiley mill. The individualized trichome fluff passes through an air classifier (Hosokawa Alpine 50ATP); which separates the “accepts” or “fine” fraction from the “rejects” or “coarse” fraction. The resultant “accepts” or “fine” fraction is greatly enriched in individualized trichomes wherein the “rejects” or “coarse” fraction is primarily chunks of stalks, and leaf elements with only a minor fraction of individualized trichomes.

A squirrel cage speed of 9000 rpm, an air pressure resistance of 10-15 mbar, and a feed rate of about 10 g/min are used on the 50 ATP. The resulting individualized trichome material (fines) is mixed with a 10% aqueous dispersion of “Texcare 4060” to add about 10% by weight “Texcare 4060” by weight of the bone dry weight of the individualized trichomes followed by slurrying the “Texcare”-treated trichomes in water at 3% consistency using a conventional repulper. This slurry is passed through a stock pipe toward another stock pipe containing eucalyptus fiber slurry.

The aqueous slurry of eucalyptus fibers is prepared at about 3% by weight using a conventional repulper. This slurry is also passed through a stock pipe toward the stock pipe containing the trichome fiber slurry.

Separately, an aqueous slurry of NSK fibers of about 3% by weight is made up using a conventional repulper making up the center layer.

The 0.2% trichome slurry is made using a third conventional repulper. This slurry and the 3% eucalyptus fiber slurry are combined in a proportion which yields 2% trichome fibers of the entire sheet at the fan pump. The wire side is the side adjacent to the forming wire and the fabric side is in contact with the drying fabric. In the three fan pump process, the composition of the center fan pump is 100% NSK, the composition of the fabric side fan pump is 100% eucalyptus, and the composition of the wire side fan pump is about 5.5% trichome fiber and 94.5% eucalyptus fiber.

These three fabric, center, and wire fan pumps are directed towards the headbox of a Fourdrinier machine.

In order to impart temporary wet strength to the finished fibrous structure, a 1% dispersion of temporary wet strengthening additive (e.g., Parez.™. 750) is prepared and is added to the NSK fiber stock pipe at a rate sufficient to deliver 0.3% temporary wet strengthening additive based on the dry weight of the NSK fibers. The absorption of the temporary wet strengthening additive is enhanced by passing the treated slurry through an in-line mixer.

The trichome and eucalyptus fiber slurry is combined and diluted with white water at the inlet of a fan pump to a consistency of about 0.15% based on the total weight of the eucalyptus and trichome fiber slurry. The NSK fibers, likewise, are diluted with white water at the inlet of a fan pump to a consistency of about 0.15% based on the total weight of the NSK fiber slurry. The eucalyptus/trichome fiber slurry and the NSK fiber slurry are both directed to a layered headbox capable of maintaining the slurries as separate streams until they are deposited onto a forming fabric on the Fourdrinier.

“DC 2310” (Dow Corning, Midland, Mich.) antifoam is dripped into the wirepit to control foam to maintain whitewater levels of 10 ppm.

The fibrous structure making machine has a layered headbox having a top chamber, a center chamber, and a bottom chamber. The eucalyptus/trichome combined fiber slurry is pumped through the top and bottom headbox chambers and, simultaneously, the NSK fiber slurry is pumped through the center headbox chamber and delivered in superposed relation onto the Fourdrinier wire to form thereon a three-layer embryonic web, of which about 70% is made up of the eucalyptus fibers and 30% is made up of the NSK fibers. Dewatering occurs through the Fourdrinier wire and is assisted by a deflector and vacuum boxes. The Fourdrinier wire is of a 5-shed, satin weave configuration having 87 machine-direction and 76 cross-machine-direction monofilaments per inch, respectively. The speed of the Fourdrinier wire is about 750 fpm (feet per minute).

The embryonic wet web is transferred from the Fourdrinier wire, at a fiber consistency of about 15% at the point of transfer, to a patterned drying fabric. The speed of the patterned drying fabric is the same as the speed of the Fourdrinier wire. The drying fabric is designed to yield a pattern densified tissue with discontinuous low-density deflected areas arranged within a continuous network of high density (knuckle) areas. This drying fabric is formed by casting an impervious resin surface onto a fiber mesh supporting fabric. The supporting fabric is a 98×62 filament, dual layer mesh. The thickness of the resin cast is about 12 mils above the supporting fabric. A suitable process for making the patterned drying fabric is described in published application US 2004/0084167 A1.

Further de-watering is accomplished by vacuum assisted drainage until the web has a fiber consistency of about 30%.

While remaining in contact with the patterned drying fabric, the web is pre-dried by air blow-through pre-dryers to a fiber consistency of about 65% by weight.

After the pre-dryers, the semi-dry web is transferred to the Yankee dryer and adhered to the surface of the Yankee dryer with a sprayed creping adhesive. The creping adhesive is an aqueous dispersion with the actives consisting of about 22% polyvinyl alcohol, about 11% CREPETROL A3025, and about 67% CREPETROL R6390. CREPETROL A3025 and CREPETROL R6390 are commercially available from Hercules Incorporated of Wilmington, Del. The creping adhesive is delivered to the Yankee surface at a rate of about 0.15% adhesive solids based on the dry weight of the web. The fiber consistency is increased to about 97% before the web is dry creped from the Yankee with a doctor blade.

The creping/drying section is set and ran as exemplified above. The total dry tensile strength was measured as described supra. The resulting total dry tensile strength for the fibrous structure product having 2% (by weight) trichomes in a first layer is 374 g/cm (or 14% increased over the comparable non-trichome ply discussed supra).

All documents cited in the Detailed Description of the Invention are, in relevant part, incorporated herein by reference; the citation of any document is not to be construed as an admission that it is prior art with respect to the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A layered fibrous structure comprising: a machine direction, cross machine direction, and Z-direction, consumer side, a first layer in the Z-direction and a second layer in the Z-direction wherein the first layer comprises a plurality of trichomes wherein the trichomes comprise greater than from about 0.1% of the fibrous structure by weight.

2. The fibrous structure of claim 1 wherein the first layer comprises from about 0.1% to about 15% of trichomes by weight of the fibrous structure.

3. The fibrous structure of claim 2 wherein the first layer comprises from about 1% to about 8% of trichomes by weight of the fibrous structure.

4. The fibrous structure of claim 3 wherein the first layer comprises from about 2% to about 4% of trichomes by weight of the fibrous structure.

5. The fibrous structure according to claim 1 wherein the first layer comprises from about 5% to about 50% of the fibrous structure in the Z-direction.

6. The fibrous structure according to claim 1 wherein the first layer in on the consumer side of the fibrous structure.

7. The fibrous structure according to claim 1 further comprising a third layer in the Z-direction.

8. A layered fibrous structure comprising a machine direction, cross machine direction, and Z-direction, consumer side, a first layer in the Z-direction and a second layer in the Z-direction wherein the first layer comprises a plurality of trichomes wherein the trichomes comprise greater than about 0.1% of the fibrous structure by weight, the fibrous structure further comprising a total dry tensile strength, the total dry tensile strength being at least about 10% greater than the total dry tensile strength of a comparable non-trichome ply.

9. The ply of fibrous structure according to claim 8 wherein the ply of fibrous structure comprises a total dry tensile strength of from about 10% to about 40% greater than the total dry tensile strength of a comparable non-trichome ply.

10. The ply of fibrous structure according to claim 9 wherein the ply of fibrous structure comprises a total dry tensile strength of from about 15% to about 30% greater than the total dry tensile strength of a comparable non-trichome ply.

11. The fibrous structure according to claim 8 wherein the first layer is on the consumer side.

12. The fibrous structure paper according to claim 8 further comprising a third layer in the Z-direction.

13. A method for making a layered fibrous structure comprising two or more layers wherein at least one of the layers comprises a plurality of trichomes comprising the steps of:
a) preparing a first fiber furnish comprising a plurality of trichomes;
b) preparing a second fiber furnish;
c) combining the first fiber furnish and the second fiber furnish to provide a third fiber furnish;
d) depositing the third fiber furnish on a forming surface to form an embryonic fibrous web; and
e) drying the embryonic fibrous web.

14. The method according to claim 13 further comprising the steps of:
f) preparing a fourth fiber furnish;
g) preparing a fifth fiber furnish; and
h) depositing the fourth and fifth fiber furnishes on the forming surface prior to drying to form an embryonic fibrous web.

15. The method according to claim 16 wherein the third fiber furnish forms a separate layer from the one or more other layers.

16. The method according to claim 13 further comprising the step of:
i) drying the embryonic fibrous web on a Yankee dryer to form the fibrous structure.

17. The method according to claim 16 further comprising the step of:
j) creping the fibrous structure off the Yankee dryer using a creping blade.

18. The method according to claim 17 wherein the creping blade is positioned in relationship to the Yankee dryer at a blade bevel angle of greater than about 65 degrees.

19. The method according to claim 18 wherein the blade bevel angle is from about 65 degrees to about 90 degrees.

20. The method according to claim 13 wherein the fibrous structure further comprises a total dry tensile strength, the total dry tensile strength being at least about 10% greater than the total dry tensile strength of a comparable non-trichome fibrous structure.
